(19) United States
(12) Patent Application Publication
(10) Pub. No.: US 2025/0283183 A1
(43) Pub. Date: Sep. 11, 2025

(54) NESTED-CLICKSEQ FOR TARGETED AMPLICON AND PROVIRUS/TRANSGENE JUNCTION SEQUENCING

(71) Applicant: Board of Regents, The University of Texas System, Austin, TX (US)

(72) Inventors: Yiyang Zhou, Galveston, TX (US); Andrew Routh, Galveston, TX (US)

(21) Appl. No.: 19/071,030

(22) Filed: Mar. 5, 2025

Related U.S. Application Data
(60) Provisional application No. 63/561,905, filed on Mar. 6, 2024.

(51) Int. Cl. C12Q 1/70; C12Q 1/6874

(52) U.S. Cl. CPC C12Q 1/702; C12Q 1/6874

(57) ABSTRACT

Provided herein are composition and methods for characterizing target nucleic acid sequences comprising: contacting a target nucleic with first primers comprising first sequences complementary to a target of interest; performing a reverse transcription reaction with a nucleic acid, the first primers and one of more terminating nucleotides, dNTPs and a reverse transcriptase to form azido-terminated cDNAs of various lengths; chemically ligating a functionalized 5′ first adaptor to the terminated cDNAs; and amplifying the chemically-ligated terminated cDNA into an amplification product using second primers comprising second sequences complementary to the target of interest, wherein the one or more second primers comprises a sequence nested between the one or more first primers and a 3′ end of the azido-terminated cDNA molecule, wherein the cDNA comprises sequences from the target of interest.

### CROSS-REFERENCE TO RELATED APPLICATIONS

This application claims priority to U.S. Provisional Application Ser. No. 63/561,905, filed Mar. 6, 2024, entitled “Nested-ClickSeq for Targeted Amplicon and Provirus/Transgene Junction Sequencing”, which is hereby incorporated by reference in its entirety.

### STATEMENT OF FEDERALLY FUNDED RESEARCH

This invention was made with government support under AI170855 awarded by the National Institutes of Health. The government has certain rights in the invention.

### TECHNICAL FIELD

The present invention relates in general to the field of simple protocols for provirus/transgene characterization, and more particularly, to a nested-ClickSeq for targeted amplicon and provirus/transgene junction sequencing.

### INCORPORATION-BY-REFERENCE OF MATERIALS FILED ON COMPACT DISC

The application contains a Sequence Listing which has been submitted electronically in .XML format and is hereby incorporated by reference in its entirety. Said .XML copy, created on Mar. 5, 2025, is named “UTMB1076.xml” and is 57,173 bytes in size. The sequence listing contained in this .XML file is part of the specification and is hereby incorporated by reference herein in its entirety.

### BACKGROUND

Without limiting the scope of the disclosure, its background is described in connection with provirus/transgene characterization.

Whole genome sequencing (WGS) of virus isolates from clinical or field samples is a critical aspect of epidemiology and surveillance. Particularly, during the course of an outbreak or pandemic, WGS allows researchers and doctors to trace the origin and transmission of individual species/strains of viruses between their hosts. It also allows researchers to determine whether and how the virus is evolving or adapting to its host over time. There are many examples of large scale whole viral genome sequencing efforts including during regular flu seasons, Ebola outbreaks, and the SARS-COV-2 pandemic.

Some viruses are able to integrate their nucleic acid genome into their host's genome. This is, for example, an essential step in the lifecycle of HIV, where the RNA genome is reverse-transcribed and the DNA pro-virus is integrated into the host DNA of the infected CD4+ cells (e.g., T cells). This integration step results in a viral ‘reservoir’ within the host and effectively prevents eradication of the virus without the concomitant destruction of the host cell. In clinical settings, determining the integration site and sequence of the provirus is an important step in providing personalized care via choice of most efficacious anti-viral strategy.

These same mechanisms of viral integration are also exploited in many gene-therapy approaches and in biotechnologies that use viral (or other) vectors to integrate selected DNA sequences into the genomic DNA of the target cells. A critical step in determining the success of genetic engineering approaches is to characterize the site of the integrant gene, which is often performed through directed polymerase chain reaction (PCR) approaches or high-throughput next-generation sequencing approaches. Next-Generation Sequencing (NGS) is often required when the integration site is not known, or when non-specific, off-target integration sites must be found/determined. Indeed, viral integration sites in real HIV infections can be diverse and complex, requiring equally complex methods to detect them.

Typically, WGS is achieved through non-targeted (random) next-generation sequencing of virus isolates amplified in cell culture or directly from patient samples. However, in cases were virus isolation and amplification are not feasible, the virus must be directly sequenced from its source. This is challenging however, as low viral genome copy numbers preclude the use of randomly-primed methods due to an inherent lack of sensitivity, thus, necessitating a targeted approached followed by PCR amplification of the virus in question. Amplification can be achieved in many ways, such as routine PCR, loop-mediated isothermal amplification (LAMP), and other iso-thermal whole genome amplification reactions. Generally, all these methods of amplification require knowledge of the virus genome sequence and require the ability to select pairs of nucleic acid oligo primers that anneal to the target genome. Furthermore, these amplification and targeting steps must be followed by additional stages to add the final platform-specific adaptors required (e.g., ILLUMINA®). In the case of sequencing integrated proviruses or transgenes, amplicon approaches that reveal the integration site are not possible because one of the primer pairs would need to anneal to the site in the host genome flanking the proviral sequencing, which (by definition) is not known a priori.

Therefore, there is an existing need for a simplified and routine methods that allows for sensitive sequencing of low-abundance targets and provirus/transgene integration sites.

### SUMMARY

As embodied and broadly described herein, an aspect of the present disclosure relates to a method for cDNA synthesis of a target nucleic acid sequence: contacting a target nucleic acid of RNA or DNA with one or more first primers comprising one or more first sequences complementary to a target of interest; performing a reverse transcription reaction with a target nucleic acid, the one or more first primers and one of more terminating nucleotides selected from modified-deoxyGTP, modified-deoxyTTP, modified-deoxyUTP, modified-deoxyCTP and modified-deoxyATP, dNTPs and a reverse transcriptase to form azido-terminated cDNAs of various lengths; chemically ligating a functionalized 5′ first adaptor to the terminated cDNAs; and amplifying the chemically-ligated terminated cDNA into an amplification product using one or more second primers comprising one or more second sequences complementary to the target of interest and optionally a second adaptor sequence, wherein the one or more second primers comprises a sequence nested between the one or more first primers and a 3′ end of the azido-terminated cDNA molecule, wherein the cDNA comprises sequences from the target of interest. In one aspect, wherein the target nucleic acid is a host sequence and further comprising sequencing the host sequence to determine a location of the target of interest. In another aspect, the cDNA further comprises additional sequences downstream of the one or more first primers. In another aspect, the target of interest is selected from natural or synthetic nucleic acid sequences, either RNA or DNA, such as viral, bacterial, fungal, archaeal, plant or metazoan nucleic acid sequences, including their genome sequences or transcripts thereof, which potentially bear mutations, translocations, fusions, insertions, deletions, integrated proviruses, transposons or transgenes, or be integrated into another nucleic acid. In another aspect, the modified-deoxyGTP, modified-deoxyCTP, modified-deoxyTTP, modified-deoxyUTP, and modified-deoxyATP are 2′- or 3′-azido-nucleotides selected from azido-GTP (AzGTP), 2′- or 3′-azido-CTP (AzCTP), 2′- or 3′-azido-ATP (AzATP), 2′- or 3′-azido-TTP (AzUTP), and 2′- or 3′-azido-TTP (AzTTP), or propargyl-GTP, propargyl-TTP, propargyl-UTP, propargyl-CTP, or propargyl-ATP. In another aspect, a ratio of the 2′- or 3′-azido-nucleotides (e.g. AzGTP, AzCTP, AzTTP, AzUTP, and AzATP) to dNTPs is 1:1000, 1:900, 1:800, 1:750, 1:700, 1:600, 1:500, 1:400, 1:300, 1:250, 1:200, 1:100, 1:90, 1:80, 1:75, 1:70, 1:60, 1:50, 1:40, 1:30, 1:25, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 1:0.5. In another aspect, a ratio of AzTTP or AzUTP:AzGTP:AzCTP:AzATP is w:x:y:z, wherein w is 0.1-10.0, x is 0.1-10.0, y is 0.1-10.0, and z is 0.1-10.0. In another aspect, the method further comprises purifying the cDNA away from the 3′-azido-nucleotides after the reverse transcription and before the amplification step. In another aspect, the method further comprises a purification step after the chemical ligation, the amplification, or both, is by column separation, magnetic bead separation, streptavidin magnetic bead wash, precipitation, solute sequestration, or surface immobilization. In another aspect, the method further comprises separating the amplification products according to their length, by gel electrophoresis, polyacrylamide gel electrophoresis, capillary electrophoresis, pulsed-field electrophoresis, agarose gel electrophoresis, PAGE, Solid Phase Reversible Immobilization (SPRI) size fractionation, selective precipitation, or pulsed-field capillary electrophoresis. In another aspect, the step of chemically ligating is defined further as click-ligating an alkyne-functionalized 5′ adaptor to the azido-terminated cDNA or an azide-functionalized 5′ adaptor to a propargyl-terminated cDNA is defined further as taking place in a buffered solution comprising: a solvent; with or without one or more metal catalysts selected from copper and ruthenium; a chelating ligand; and an accelerant. In another aspect, the method further comprises purifying the chemically ligated-cDNA-adaptor away from unligated adaptors before an amplification step. In another aspect, the purification step is by column separation, magnetic bead separation, selective precipitation, surface immobilization, or streptavidin magnetic bead wash. In another aspect, the reverse transcription is performed by a reverse transcriptase (RT) derived from Avian Myeloblastosis Virus Reverse Transcriptase, Respiratory Syncytial Virus Reverse Transcriptase, Moloney

Murine Leukemia Virus Reverse Transcriptase, Human Immunodeficiency Virus Reverse Transcriptase, Equine Infectious Anemia Virus Reverse Transcriptase, Rous-Associated Virus 2 Reverse Transcriptase, Avian Sarcoma Leukosis Virus Reverse Transcriptase, RNaseH (−) Reverse Transcriptase, SuperScript II Reverse Transcriptase, SuperScript III Reverse Transcriptase, SuperScript IV Reverse Transcriptase, thermostable group II intron reverse transcriptases (TGIRT), Therminator DNA Polymerase, or ThermoScript Reverse Transcriptase, wherein an RNase H activity of these RTs is present, reduced or not present. In another aspect, the method further comprises determining an identity or sequence of the amplification products by an automated process on a chip, Sanger sequencing, Maxam-Gilbert sequencing, dye terminator sequencing, sequencing by synthesis, pyrosequencing, microarray hybridization, next-generation sequencing methods, next-next-generation sequencing, ion semiconductor sequencing, polony sequencing, sequencing by ligation, DNA nanoball sequencing, or single molecule sequencing. In another aspect, the genomic nucleic acid is from a biological fluid, biopsy, cells, or tissue. In another aspect, the first, the second, and one or more additional primers are present in the same orientation, direction, or strandedness. In another aspect, a selectivity of the reverse transcription, the amplification, or both, is increased by using trehalose, betaine, tetramethylammonium chloride, tetramethylammonium oxalate, formamide and oligo-blockers, or dimethylsulfoxide during a polymerase chain reaction, to reduce an occurrence of mispriming. In another aspect, a DNA polymerase used for the amplifying step is Taq DNA polymerase, Tfl DNA polymerase, a Taq DNA polymerase, a Klenow fragment, Sequenase or Klentaq an enzyme with proof reading activity, preferably selected from the PFU, Ultma, Vent, Deep Vent, PWO, or Tli polymerase. In another aspect, the method further comprises purifying a PCR product from the step of amplifying the clicked-cDNA with a column separation, magnetic bead separation, selective precipitation, surface immobilization or streptavidin magnetic bead wash. In another aspect, an alkyne-functionalized, or azide-functionalized, 5′ adaptor comprises all nucleotides NNNNNN, N₀₋₂₄, a, Unique Molecular Index, Barcoding Index, semi-random primers, or a specific template primer sequence, or the adapter comprises a unique sequence. In another aspect, the terminating deoxynucleotides contain a chemically reactive functional group at either the 3′ or 2′ site of the ribose ring selected from azido-nucleotides (AzGTP, AzCTP, AzTTP, AzUTP and AzATP), propargyl-nucleotides (propargyl-GTP, propargyl-CTP, propargyl-TTP, propargyl-UTP, and propargyl-ATP), amino-nucleotides (AmGTP, AmCTP, AmTTP, AmUTP, and AmATP), or halogenated nucleotides (Hal-GTP, Hal-CTP, Hal-TTP, Hal-UTP, and Hal-ATP).

As embodied and broadly described herein, an aspect of the present disclosure relates to a kit for nested-ClickSeq comprising: one or more vials comprising: one or more first primers comprising one or more first sequences complementary to a target of interest; terminating nucleotides of modified-deoxyGTP, modified-deoxyTTP, modified-deoxyUTP, modified-deoxyCTP, and modified-deoxyATP, and dNTPs; one or more vials comprising a reverse transcriptase; a cDNA fragment isolating kit; one or more vials comprising components for chemically ligating a functionalized 5′ first adaptor to the cDNA; a DNA amplification kit for amplifying the chemically-ligated cDNA into an amplification product; a functionalized 5′ first adaptor for chemically ligating to a terminated cDNAs; and one or more second primers comprising one or more second sequences complementary to the target of interest and optionally a second adaptor sequence, wherein the one or more second primers comprises a sequence nested between the one or more first primers and a 3′ end of the azido-terminated cDNA molecule, wherein the cDNA comprises sequences from the target of interest, wherein the one or more second primers amplify a chemically-ligated terminated cDNA; and instructions for amplification of a transposable element inserted in a host genome without fragmentation or enzymatic ligation. In one aspect, the terminating modified-deoxyGTP, modified-deoxyCTP, modified-deoxyTTP, modified-deoxyUTP, and modified-deoxyATP are 2′- or 3′-azido-nucleotides selected from azido-GTP (AzGTP), 2′- or 3′-azido-CTP (AzCTP), 2′- or 3′-azido-ATP (AzATP), 2′- or 3′-azido-TTP (AzTTP), and 2′- or 3′-azido-UTP (AzUTP), or propargyl-GTP, propargyl-TTP, propargyl-UTP, propargyl-CTP, or propargyl-ATP. In another aspect, an alkyne or azide modified oligo ‘click’ reaction is a hexanyl-oligo or azide-oligo. In another aspect, a ratio of the 2′- or 3′-azido-nucleotides (AzGTP, AzCTP, AzTTP, and AzATP) to dNTPs is 1:1000, 1:900, 1:800, 1:750, 1:700, 1:600, 1:500, 1:400, 1:300, 1:250, 1:200, 1:100, 1:90, 1:80, 1:75, 1:70, 1:60, 1:50, 1:40, 1:30, 1:25, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 1:0.5. In another aspect, a ratio of AzTTP or AzUTP:AzGTP:AzCTP:AzATP is w:x:y:z, wherein w is 0.1-10.0, x is 0.1-10.0, y is 0.1-10.0, and z is 0.1-10.0. In another aspect, the kit further comprises a cDNA purification kit for purifying a cDNA away from 2′ or 3′-azido-nucleotides after reverse transcription and before the amplifying step selected from a column separation kit, magnetic bead separation kit, streptavidin magnetic bead kit, precipitation, solute sequestration, or surface immobilization. In another aspect, the kit further comprises a clicked-cDNA-adaptor purification kit for separating clicked-cDNA-adaptors away from unligated alkyne-functionalized 5′ adaptors before the amplifying step selected from a column separation kit, magnetic bead separation kit, streptavidin magnetic bead kit, precipitation, solute sequestration, or surface immobilization. In another aspect, the click-ligating components comprise: an alkyne-functionalized 5′ adaptor to the azido-terminated cDNA or an azido-terminated cDNA; a buffered solution comprising: a solvent mix comprising DMSO, water, and ethanol; metal catalysts selected from copper and ruthenium; a chelating ligand; and an accelerant. In another aspect, the reverse transcriptase (RT) is an RT derived from Avian Myeloblastosis Virus Reverse Transcriptase, Respiratory Syncytial Virus Reverse Transcriptase, Moloney Murine Leukemia Virus Reverse Transcriptase, Human Immunodeficiency Virus Reverse Transcriptase, Equine Infectious Anemia Virus Reverse Transcriptase, Rous-Associated Virus 2 Reverse Transcriptase, Avian Sarcoma Leukosis Virus Reverse Transcriptase, RNaseH (−) Reverse Transcriptase, SuperScript II Reverse Transcriptase, SuperScript III Reverse Transcriptase, SuperScript IV Reverse Transcriptase, thermostable group II intron reverse transcriptases (TGIRT), Therminator DNA Polymerase, or ThermoScript Reverse Transcriptase, wherein an RNase H activity of the RTs is present, reduced or not present. In another aspect, the target of interest is selected from natural or synthetic nucleic acid sequences, either RNA or DNA, such as viral, bacterial, fungal, archaeal, plant or metazoan nucleic acid sequences, including their genome sequences or transcripts thereof, which potentially bear mutations, translocations, fusions, insertions, deletions, integrated proviruses, transposons or transgenes, or be integrated into another nucleic acid. In another aspect, a selectivity of the reverse transcription and/or amplification with a polymerase chain reaction or loop-mediated isothermal amplification, is increased by using trehalose, betaine, tetramethylammonium chloride, tetramethylammonium oxalate, formamide and oligo-blockers, or dimethylsulfoxide during the polymerase chain reaction, to reduce mispriming. In another aspect, the kit further comprises a sequencing kit determining an identity or sequence of the amplification products by an automated process on a chip, Sanger sequencing, Maxam-Gilbert sequencing, dye terminator sequencing, sequencing by synthesis, pyrosequencing, microarray hybridization, next-generation sequencing methods, next-next-generation sequencing, ion semiconductor sequencing, polony sequencing, sequencing by ligation, DNA nanoball sequencing, or single molecule sequencing. In another aspect, the kit further comprises a DNA polymerase used for the amplifying reaction is Taq DNA polymerase, Tfl DNA polymerase, Taq DNA polymerase, Klenow fragment, Sequenase or Klentaq with proof reading activity selected from PFU, Ultma, Vent, Deep Vent, PWO, or Tli polymerases. In another aspect, the kit further comprises a kit for purifying a PCR product from the step of amplifying a clicked-nested-cDNA step with a column separation, magnetic bead separation, selective precipitation, surface immobilization or streptavidin magnetic bead wash. In another aspect, the alkyne-functionalized 5′ adaptor comprises all nucleotides NNNNNN, N₀₋₁₂, a Unique Molecular Index, Barcoding Index, semi-random primers, or a specific template primer sequence, or the adapter comprises a unique sequence. In another aspect, the first, the second, and optionally one or more additional nested primers are present in a same orientation, direction, or strandedness.

As embodied and broadly described herein, an aspect of the present disclosure relates to a method for determining a location of a target nucleic acid sequence: contacting a genomic DNA with one or more first primer comprising a first sequence complementary to a target nucleic acid sequence; performing a reverse transcription reaction with a genomic nucleic acid, the one or more first primers and one of more terminating nucleotides selected from modified-deoxyGTP, modified-deoxyCTP, modified-deoxyTTP, modified-deoxyATP, dNTPs, and a reverse transcriptase to form terminated cDNAs of various lengths; chemically ligating a functionalized 5′ first adaptor to the terminated cDNAs; amplifying the chemically-ligated terminated cDNA into an amplification product using one or more second primers comprising to a second sequence complementary to the target of interest, wherein the one or more second primers comprises a sequence nested between the one or more first primers and a 3′ end of the azido-terminated cDNA molecule, wherein the cDNA comprises sequences from the target of interest; and sequencing the host sequences and comparing to a genome to determine a location of an insertion of the target of interest.

### BRIEF DESCRIPTION OF THE DRAWINGS

For a more complete understanding of the features and advantages of the present disclosure, reference is now made to the detailed description of the disclosure along with the accompanying figures and in which:

FIG. 1A shows the steps in the ClickSeq method of the prior art, which has been previously described and validated by the present inventors.

FIG. 1B shows the ‘Nested-ClickSeq’ approach of the present invention that specifically amplifies the target of interest using a template-specific primer in the reverse transcriptase (RT) step of a ‘ClickSeq’ protocol, another template-specific primer is used in the PCR step of the ‘ClickSeq’ protocol.

FIG. 2 shows the primer maps of the 5′ LTR of a transposable element.

FIG. 3 shows the primer maps of the 3′LTR of a transposable element.

FIG. 4 shows the results from a Nested-ClickSeq synthesis, which generated robust libraries for the J-Lat derived DNA but not for the negative-control Jurkat-derived DNA (that lacks the HIV provirus).

FIG. 5 is a graph that summarizes the results obtained using the Nested-ClickSeq synthesis on J-Lat cell line with documented HIV pro-virus genome integrations (positive sample). Nested-ClickSeq successfully revealed 1821-12766 virus integration events per one million of reads, from 4 primers.

FIG. 6 is a graph that summarizes the results obtained from negative samples. From negative samples (Jurkat cell line without HIV pro-virus insertion), Nested-ClickSeq also exhibited equal or better specificity than regular non-nested ClickSeq.

### DETAILED DESCRIPTION

While the making and using of various aspects of the present disclosure are discussed in detail below, it should be appreciated that the present disclosure provides many applicable inventive concepts that can be embodied in a wide variety of specific contexts. The specific aspects discussed herein are merely illustrative of specific ways to make and use the disclosure and do not delimit the scope of the disclosure.

To facilitate the understanding of this disclosure, a number of terms are defined below. Terms defined herein have meanings as commonly understood by a person of ordinary skill in the areas relevant to the present disclosure. Terms such as “a”, “an” and “the” are not intended to refer to only a singular entity, but include the general class of which a specific example may be used for illustration. The terminology herein is used to describe specific aspects of the disclosure, but their usage does not delimit the disclosure, except as outlined in the claims.

The present invention is a novel ‘Nested-ClickSeq’ approach that specifically amplifies a target of interest. In addition to using a template-specific primer in the RT step of a ‘ClickSeq’ protocol, another template-specific primer is used in the PCR step of the ‘ClickSeq’ protocol. Thus, the PCR step now provides a second layer of template selection that: 1) avoids amplification of cDNA molecules that are the product of non-specific/off-target selection from the RT-primer (since these would not contain the nested-target annealing sequences; 2) allows for multiple rounds of selection with each PCR cycle (rather than the single selection step used in Tiled-ClickSeq). The same click-ligation adaptors and cognate primers for these adaptors are still used as before, however, the new method retains the unique advantages and properties of the ClickSeq-based platform, while ‘boosting’ the specificity and sensitivity of the approach. Although multiple template-specific primers may be required and used to generate each sequence amplicon, the specific ClickSeq-based strategy uses nested primers such that each of these primers are present in the same orientation/direction/strandedness as one another. The present invention overcomes two critical problems in the prior art.
Problem 1: Complexity of assay: A plethora of complex methods have been envisaged that enrich for the provirus/host genome junction site without the need to know the flanking host sequence. These include hybrid capture probes, host depletion methods and restriction digests. All of these methods rely on host DNA fragmentation prior to the enrichment. Further, amplification is typically achieved through the ligation of DNA adaptors onto DNA fragments followed by PCR-amplification of the target amplicons of interest using primer cognate to the ligated adaptors and the provirus/transgene of interest. All of these methods are technically complex and there is no straight-forward product on the market that provides the reagents and protocols to perform these assays. As a result, they are often the remit of focused/specialized genomics labs/cores, that rely on expert technicians.
Problem 2: Sensitivity of assay: In principle, provirus/transgene junctions can be sequenced simply by deep-sequencing of the nucleic acids of interest. Random-primed methods would capture all templates. This is highly inefficient however, as all nucleic acids would be captured, rather than just the provirus/transgene of interest. In ‘Tiled-ClickSeq” (Jaworski et al, eLife, (2021)), the present inventors described a method to achieve whole genome sequencing (WGS) of a virus isolate, where multiple tiled primers were use spaced evenly along the virus genome, each only targeting one annealing site and without the need to design corresponding paired PCR primers. This simplified the assay design, but importantly removes that constraint and limitations ordinary imposed in paired-primer amplicon approaches. The method accurately captures full-length viral genomes as well as the recombination RNA species present (such as sub-genomic mRNAs). Importantly, such recombinant species can also include recombination between a virus of interest and the host genetic material (as occurs during a virus or transgene integration event). As such, Tiled-ClickSeq might provide a simple method for transgene/provirus integration site sequencing.

To the inventors' knowledge, Tiled-ClickSeq is the only single-primer-per-amplicon tiled-sequencing approach described or commercially developed to date. However, a limitation of this approach is when the target is of very low abundance and the tiled-primers non-specifically anneal to off-target nucleic adds. This results in off-target sequencing and reduces the specificity of the capture of the target of interest. It was shown by the present inventors in Jaworski, et. al, eLife (2021) that read coverage over the SARS-COV-2 genome when using Tiled-ClickSeq began to drop at CT values of 20-25 for SARS-COV-2 in clinical settings. For integration assays, the integrated DNA provirus or transgene is necessarily at very low abundance. Since only total host DNA can be provided as a source, and only less than one integrant is likely to be present per host genome present, the amount of viral or transgene nucleic acid is thus vanishingly small. For example, 100 ngs of host DNA purified from white blood cells of a HIV-infected human corresponds to only ˜150 femtograms of target nucleic acid. Further, this is assuming that 100% of the cells carry one provirus, which is highly unlikely. In reality, >1% of the CD4+ cells will harbor an intact HIV provirus, thus reducing the target nucleic acid content to only a femtogram per 100 nanograms of purified DNA.

The present inventors have devised a method that circumvents these challenges and provides a simple protocol for provirus/transgene characterization that can be performed in a routine manner, using existing reagents. The current invention is built upon the ‘ClickSeq’ approach for Next-Generation Sequencing (NGS). Two unique features of ClickSeq include the requirement of only one template-specific primer per amplicon, and that the 3′end of an amplified cDNA segment is generated stochastically through the use of terminating 3′-azido-nucleotides that are incorporated during reverse transcription. Thereafter, a downstream primer is click-ligated onto the cDNA and so a second template-specific primer is not required. Thus, the provirus DNA and the exact integration site can be amplified in a straightforward manner using PCR with one-template-specific primer, and one click-adaptor specific primer.

The ‘Nested-ClickSeq’ approach specifically amplifies the target of interest using a template-specific primer in the reverse transcriptase (RT) step of a ‘ClickSeq’ protocol, while another template-specific primer is used in the PCR step of the ‘ClickSeq’ protocol. Thus, the PCR step now provides a second layer of template selection that: (1) avoids cDNA molecules that are the product of non-specific/off-target selection from the RT-primer (since these would not contain the nested-target annealing sequences; and/or (2) allows for multiple rounds of selection with each PCR cycle (rather than the single selection step used in Tiled-ClickSeq). The same click-ligation adaptors and cognate primers for these adaptors are still used as before (that is, retains the unique advantages and properties of the ClickSeq-based platform), while ‘boosting’ the specificity and sensitivity of the approach. While the present invention uses multiple template-specific primers that are used to generate each sequence amplicon, the specific ClickSeq-based strategy using nested primers means that each of these nested primers are present in the same orientation/direction/strandedness as one another.

While seemingly straightforward, this required a re-design of the primer/adaptor sequences used in the protocol, described below, to maintain compatibility with current sequencing platforms (e.g., NANOPORE® or ILLUMINA®). It was found that this approach constitutes an important improvement on the existing approaches to sequence DNA proviruses, as demonstrated by a new capacity to capture the integration site of the HIV provirus in human DNA in a robust manner.

While described herein for using nested-ClickSeq to amplifying HIV provirus integration sites, as a non-limiting example, the process of the present invention also provides flexibility in that a random primer, a mixture of multiple tiled-primers, or a generic primer (such as an oligo-dT primer), or any composition thereof, can be used during the RT step. This provides flexibility in the types of templates that are initially reverse-transcribed and the degree of selectivity during this RT stage. The subsequent click-ligated cDNA can still be amplified using a single, or multiple, nested-PCR primers, thus still providing for multiple additional rounds of selection with each PCR cycle, again, without the need for pairs of primers spanning pre-defined amplicons in the PCR stage. The combinations of template-specific primers used in either or both the RT and PCR steps can be chosen and optimized by the practitioner per the needs of the assay, the type of nucleic acid or sample under study, and the information required by the practitioner. Since the RT primer need not have any sequencing platform specific adaptor or sequence present (since these are provided in the click-ligation step and in the PCR step), any pre-existing primer can be used in the RT step and remain compatible with the downstream protocol. This allows practitioners to take advantage of existing stock of reagents and/or RT primers designed for unrelated purposes (e.g., for qRT-PCR).

The Nested-ClickSeq approach has many additional practical, technical and scientific advantages/values. For example, with the Nested-ClickSeq approach, the same validated primer set can be used for any NGS platform, regardless of the length of fragments that are required for sequencing. For example, it is possible to adjust the 3′azido: 3′oxy deoxynucleotide mix ratio by diluting the nucleotide mix with dNTPs in order to generate amplicons of increased lengths. This greatly improves flexibility of the pipeline and obviates the need to redesign tiled primers per platform.

As used herein, the term “barcode” refers to a nucleic acid molecule having a sequence that can serve, e.g., as an identifier of the molecule (molecular barcode), identifier of the partition (partition barcode), or an identifier of the sample (sample barcode or sample index).

As used herein, the terms “detect,” “detecting,” or “detection” refer to determining the existence or presence of one or more “target nucleic acids”. Non-limiting examples of target nucleic acid sequences include (in both DNA and RNA form), e.g., nucleic acids having targeted mutations, translocations, fusions, insertions, deletions, transposons, viral sequences, whole virus genomes, bacterial or fungal genomes, microsatellites, host genomic sequences, proviruses, RNA or DNA virus genomes or their expressed viral transcripts and sub-genomic RNAs, host mRNAs, synthetic/designed nucleic acid sequences, junctions of the above examples once integrated into another nucleic acid sequence or genome, gene-gene or mRNA-mRNA fusion products such as those arise during oncogenic transformation, and various combinations of nucleic acid thereof, or other nucleic-acid markers in a sample.

As used herein, the term “sequencing” refers to techniques and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequence determination can be obtained using, such as, without limitation, a sequencing system by ILLUMINA®, Pacific Biosciences (PACBIO®), OXFORD NANOPORE®, or Life Technologies (ION TORRENT®). Sequencing may be performed using nucleic acid amplification, polymerase chain reaction (PCR) (e.g., digital PCR, quantitative PCR, or real time PCR), or isothermal amplification.

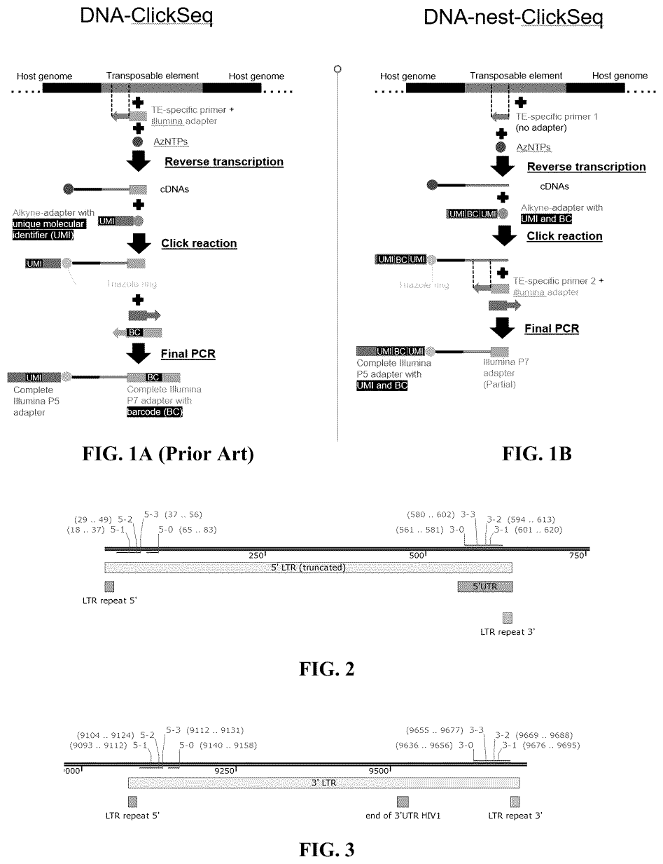

FIG. 1A shows the steps in the ClickSeq method of the prior art, which has been well-described and validated by the present inventors. Briefly, the primer used in reverse-transcription step is contains 6 random nucleotides (6N) at the 3′ end, and a partial i7 Illumina adaptor on the 5′ end (p7). The Poly (A)-ClickSeq, and Tiled-ClickSeq approaches are similar, but the RT primer contains an oligo-dT tract or a template-specific sequence in place of the 6N random nucleotides (respectively).

After reverse transcription in the presence of AzNTPs, azido-terminated cDNAs are obtained, which are ‘click-ligated’ onto ‘click adaptors’ containing, e.g., a complete i5 Illumina adaptor (15). Thus, the cDNA product has a partial i7, followed by the template cDNA, followed by the entire i5 sequence. The remaining i7 sequence (which includes the sample-specific barcode) is then added in a PCR step that uses primers targeting the i5 and partial i7. Since the PCR step only targets the i5 and i7 adaptors, there is no further template targeting, and thus no further enrichment of a species of interest.

If a template specific primer were used in the PCR step, it would anneal to the cDNA tract, and thus the PCR product would lack either the i5 or p7 sequence. Therefore, to target the cDNA in the PCR step, the PCR primers must also have the i7 or i5 adaptors present, which would constitute an excessively large primer (80-90 nts in length). Such primers would be expensive and would also perform poorly. To overcome this, there are two options for using shorter primers: (1) a template specific primer can be used in the PCR (only 20-30 nts long). This would result in no i7 sequence in the PCR product. However, an additional ligation step could be used to add this (as is often performed when using ‘stubby adaptors’). (2) A template specific primer with the same p7 sequence as used for the Tiled-ClickSeq RT reaction could be used (40-50 nts long). However, the remaining i7 sequence and barcode would have to be added (again) in another ligation reaction, and/or a second PCR reaction to fill-in the remaining required nucleotides.

While feasible, each of these processes adds additional steps (ligation and/or PCR), which is undesirable because it adds expense, time, need for additional reagents, and they each require additional DNA clean-up/purification steps, which result in sample attrition. This obviates the simplicity and efficiency desired and inherent to ‘ClickSeq’ and may also introduce artifacts, such as unwanted sequence chimeras. Therefore, the present inventors devised an alternative strategy.

FIG. 1B shows the ‘Nested-ClickSeq’ approach of the present invention that specifically amplifies the target of interest using a template-specific primer in the reverse transcriptase (RT) step of a ‘ClickSeq’ protocol, another template-specific primer is used in the PCR step of the ‘ClickSeq’ protocol. Thus, the PCR step now provides a second layer of template selection that: (1) avoids cDNA molecules that are the product of non-specific/off-target selection from the RT-primer (since these would now contain the nested-target annealing sequences; and/or (2) allows for multiple rounds of selection with each PCR cycle (rather than the single selection step used in Tiled-ClickSeq). The same click-ligation adaptors and cognate primers for these adaptors are still used as before (that is, retains the unique advantages and properties of the ClickSeq-based platform), while ‘boosting’ the specificity and sensitivity of the approach. While the present invention uses multiple template-specific primers that are used to generate each sequence amplicon, the specific ClickSeq-based strategy using nested primers means that each of these nested primers are present in the same orientation/direction/strandedness as one another.

FIG. 1B shows one unique feature of using click-chemistry to ‘click-ligate’ sequencing adaptors is that the ssDNA oligos have no practical limit on their length or complexity. As a result, it is possible to add extra features to the click-adaptor, such as random nucleotides and/or indexing sequences at this step. This allows for removal of indexing in the p7 adaptor. As a result, a much smaller p7 adaptor can be used, containing only the nucleotides required to allow cDNA adsorption onto the Illumina flowcell, rather than the full i7 indexing sequence. Thus, the PCR reaction can comprise a PCR primer that targets the i5 click-adaptor (same as in all ClickSeq PCR reactions), and one or more PCR primers that target a gene/virus/transgene/etc. of interest with a short overhand of the i7 adaptor.

FIG. 1B illustrates how the basic ClickSeq protocol is harnessed to introduce a template-selection step in the final PCR of the NGS library preparation. There is no equivalent process described that does not require additional subsequent handling/stages. This is true whether a random primer is used in the RT-reaction, or a template-specific primer was used in the RT-reaction.

To show the applicability of the novel Tiled-ClickSeq of the present invention, 200 ng of extracted DNA from J-LAT cells were obtained (kindly provided by Dr. Haitao Hu at UTMB). These samples were subjected to the canonical ClickSeq protocol, with the exception that a gene-specific primer was used in the RT reaction without an Illumina® adapter sequence. Primers named ‘5-0’ and ‘3-0’ are the 1^{st} step RT primer specific to HIV LTR sequence:

| primer | | |
|---|---|---|
| name | sequence (5′-3′) | SEQ ID NO: |
| 5-0 | CCAATCAGGGAAGTAGCCT | 1 |
| 3-0 | TGCCCGTCTGTTGTGTGACTC | 2 |

FIGS. 2 and 3 show the primer maps, of the 5′ LTR (FIG. 2), and 3′LTR (FIG. 3), respectively.

For the click-adaptors, hexynyl-functionalized oligos were designed, but with the addition of barcodes at the 5′ end of the adaptor. These barcodes were flanked with 4 random nucleotides on each side that would perform as Unique Molecular Adaptors (UMIs). These primers were ordered from IDT. The inventors ‘click-ligated’ the long barcoded/UMI click-adaptors (one unique barcode per sample) as described above, again using the canonical 5 ClickSeq protocol for click-ligation.

| | | SEQ |
|---|---|---|
| | | ID |
| Name | Sequence | NO: |
| Hex_4N-701- | /5Hexynyl/NNN NCC GCG | 3 |
| 4N_ | GTT NNN NAG ATC GGA AGA | |
| UniRevComp | GCG TCG TGT AGG GAA AGA | |
| | GTG TAG ATC TCG GTG GTC | |
| | GCC GTA TCA TT | |
| Hex_4N-702- | /5Hexynyl/NNN NTT ATA | 4 |
| 4N_ | ACC NNN NAG ATC GGA AGA | |
| UniRevComp | GCG TCG TGT AGG GAA AGA | |
| | GTG TAG ATC TCG GTG GTC | |
| | GCC GTA TCA TT | |
| Hex_4N-703- | /5Hexynyl/NNN NGG ACT | 5 |
| 4N_ | TGG NNN NAG ATC GGA AGA | |
| UniRevComp | GCG TCG TGT AGG GAA AGA | |
| | GTG TAG ATC TCG GTG GTC | |
| | GCC GTA TCA TT | |
| Hex_4N-704- | /5Hexynyl/NNN NAA GTC | 6 |
| 4N_ | CAA NNN NAG ATC GGA AGA | |
| UniRevComp | GCG TCG TGT AGG GAA AGA | |
| | GTG TAG ATC TCG GTG GTC | |
| | GCC GTA TCA TT | |
| Hex_4N-705- | /5Hexynyl/NNN NAT CCA | 7 |
| 4N_ | CTG NNN NAG ATC GGA AGA | |
| UniRevComp | GCG TCG TGT AGG GAA AGA | |
| | GTG TAG ATC TCG GTG GTC | |
| | GCC GTA TCA TT | |
| Hex_4N-706- | /5Hexynyl/NNN NGC TTG | 8 |
| 4N_ | TCA NNN NAG ATC GGA AGA | |
| UniRevComp | GCG TCG TGT AGG GAA AGA | |
| | GTG TAG ATC TCG GTG GTC | |
| | GCC GTA TCA TT | |
| Hex_4N-707- | /5Hexynyl/NNN NCA AGC | 9 |
| 4N_ | TAG NNN NAG ATC GGA AGA | |
| UniRevComp | GCG TCG TGT AGG GAA AGA | |
| | GTG TAG ATC TCG GTG GTC | |
| | GCC GTA TCA TT | |
| Hex_4N-708- | /5Hexynyl/NNN NTG GAT | 10 |
| 4N_ | CGA NNN NAG ATC GGA AGA | |
| UniRevComp | GCG TCG TGT AGG GAA AGA | |
| | GTG TAG ATC TCG GTG GTC | |
| | GCC GTA TCA TT | |
| Hex_4N-709- | /5Hexynyl/NNN NAG TTC | 11 |
| 4N_ | AGG NNN NAG ATC GGA AGA | |
| UniRevComp | GCG TCG TGT AGG GAA AGA | |
| | GTG TAG ATC TCG GTG GTC | |
| | GCC GTA TCA TT | |
| Hex_4N-710- | /5Hexynyl/NNN NGA CCT | 12 |
| 4N_ | GAA NNN NAG ATC GGA AGA | |
| UniRevComp | GCG TCG TGT AGG GAA AGA | |
| | GTG TAG ATC TCG GTG GTC | |
| | GCC GTA TCA TT | |
| Hex_4N-711- | /5Hexynyl/NNN NTC TCT | 13 |
| 4N_ | ACT NNN NAG ATC GGA AGA | |
| UniRevComp | GCG TCG TGT AGG GAA AGA | |
| | GTG TAG ATC TCG GTG GTC | |
| | GCC GTA TCA TT | |
| Hex_4N-712- | /5Hexynyl/NNN NCT CTC | 14 |
| 4N_ | GTC NNN NAG ATC GGA AGA | |
| UniRevComp | GCG TCG TGT AGG GAA AGA | |
| | GTG TAG ATC TCG GTG GTC | |
| | GCC GTA TCA TT | |
| Hex_4N-713- | /5Hexynyl/NNN NCC AAG | 15 |
| 4N_ | TCT NNN NAG ATC GGA AGA | |
| UniRevComp | GCG TCG TGT AGG GAA AGA | |
| | GTG TAG ATC TCG GTG GTC | |
| | GCC GTA TCA TT | |
| Hex_4N-714- | /5Hexynyl/NNN NTT GGA | 16 |
| 4N_ | CTC NNN NAG ATC GGA AGA | |
| UniRevComp | GCG TCG TGT AGG GAA AGA | |
| | GTG TAG ATC TCG GTG GTC | |
| | GCC GTA TCA TT | |
| Hex_4N-715- | /5Hexynyl/NNN NGG CTT | 17 |
| 4N_ | AAG NNN NAG ATC GGA AGA | |
| UniRevComp | GCG TCG TGT AGG GAA AGA | |
| | GTG TAG ATC TCG GTGGTC | |
| | GCC GTA TCA TT | |
| Hex_4N-716- | /5Hexynyl/NNN NAA TCC | 18 |
| 4N_ | GGA NNN NAG ATC GGA AGA | |
| UniRevComp | GCG TCG TGT AGG GAA AGA | |
| | GTG TAG ATC TCG GTG GTC | |
| | GCC GTA TCA TT | |
| Hex_4N-717- | /5Hexynyl/NNN NTA ATA | 19 |
| 4N_ | CAG NNN NAG ATC GGA AGA | |
| UniRevComp | GCG TCG TGT AGG GAA AGA | |
| | GTG TAG ATC TCG GTG GTC | |
| | GCC GTA TCA TT | |
| Hex_4N-718- | /5Hexynyl/NNN NCG GCG | 20 |
| 4N_ | TGA NNN NAG ATC GGA AGA | |
| UniRevComp | GCG TCG TGT AGG GAA AGA | |
| | GTG TAG ATC TCG GTG GTC | |
| | GCC GTA TCA TT | |
| Hex_4N-719- | /5Hexynyl/NNN NAT GTA | 21 |
| 4N_ | AGT NNN NAG ATC GGA AGA | |
| UniRevComp | GCG TCG TGT AGG GAA AGA | |
| | GTG TAG ATC TCG GTG GTC | |
| | GCC GTA TCA TT | |
| Hex_4N-720- | /5Hexynyl/NNN NGC ACG | 22 |
| 4N_ | GAC NNN NAG ATC GGA AGA | |
| UniRevComp | GCG TCG TGT AGG GAA AGA | |
| | GTG TAG ATC TCG GTG GTC | |
| | GCC GTA TCA TT | |
| Hex_4N-721- | /5Hexynyl/NNN NGG TAC | 23 |
| 4N_ | CTT NNN NAG ATC GGA AGA | |
| UniRevComp | GCG TCG TGT AGG GAA AGA | |
| | GTG TAG ATC TCG GTG GTC | |
| | GCC GTA TCA TT | |
| Hex_4N-722- | /5Hexynyl/NNN NAA CGT | 24 |
| 4N_ | TCC NNN NAG ATC GGA AGA | |
| UniRevComp | GCG TCG TGT AGG GAA AGA | |
| | GTG TAG ATC TCG GTG GTC | |
| | GCC GTA TCA TT | |
| Hex_4N-723- | /5Hexynyl/NNN NGC AGA | 25 |
| 4N_ | ATT NNN NAG ATC GGA AGA | |
| UniRevComp | GCG TCG TGT AGG GAA AGA | |
| | GTG TAG ATC TCG GTG GTC | |
| | GCC GTA TCA TT | |
| Hex_4N-724- | /5Hexynyl/NNN NAT GAG | 26 |
| 4N_ | GCC NNN NAG ATC GGA AGA | |
| UniRevComp | GCG TCG TGT AGG GAA AGA | |
| | GTG TAG ATC TCG GTG GTC | |
| | GCC GTA TCA TT | |

In the final PCR step, the inventors used all canonical ClickSeq reagents, including the ‘Universal Primer Short’ but place of the i7 indexing primers usually used at this step, ‘short’ 17 primers were employed with constant regions that targeted the viral LTR. The inventors designed primers comprising the short i7 sequence and a constant region targeting a virus of interest. The constant regions comprised sequences targeting either 5′ (i.e., 5-1˜5-3) or 3′ (i.e., 3-1˜3-3) terminals of HIV LTR 20-21 nts in length and 34 nts of the minimal i7 Illumina adapter (bold).

| | | SEQ |
|---|---|---|
| primer | | ID |
| name | sequence (5′-3′) | NO: |
| 5-1 | CAAGCAGAAGACGGCATACGAGAT | 27 |
| | ATCTCTTGTCTTTTTTGGGA | |
| 5-2 | CAAGCAGAAGACGGCATACGAGAT | 28 |
| | CACAGATCAAGGATCTCTTGT | |
| 5-3 | CAAGCAGAAGACGGCATACGAGAT | 29 |
| | GTAGATCCACAGATCAAGGA | |
| 3-1 | CAAGCAGAAGACGGCATACGAGAT | 30 |
| | AGACCCTTTTAGTCAGTGTG | |
| 3-2 | CAAGCAGAAGACGGCATACGAGAT | 31 |
| | ATCCCTCAGACCCTTTTAGT | |
| 3-3 | CAAGCAGAAGACGGCATACGAGAT | 32 |
| | GGTAACTAGAGATCCCTCAG | |

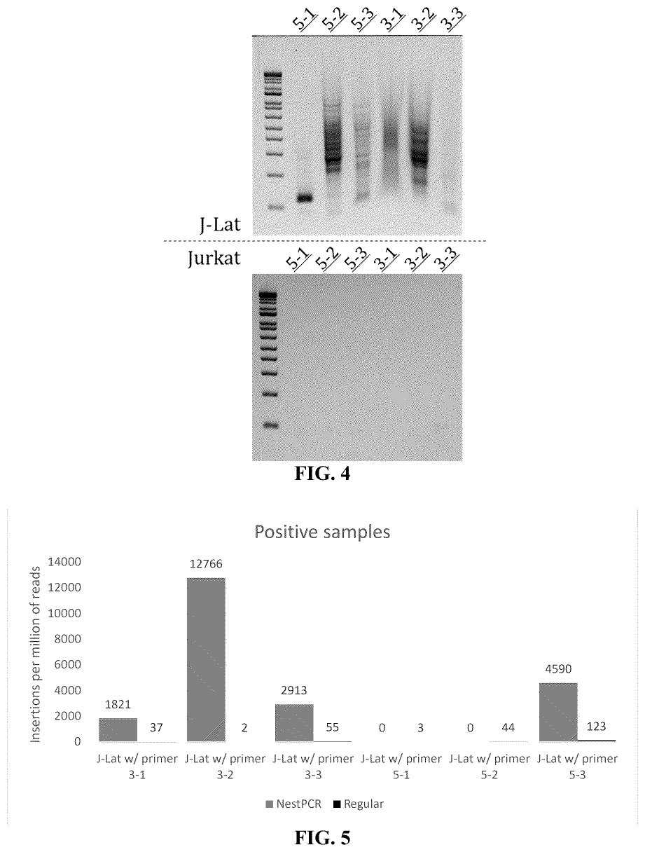

FIG. 4 shows the results from a nested-ClickSeq synthesis, which generated robust libraries for the J-Lat derived DNA but not for the negative-control Jurkat derived DNA (that lacks the HIV provirus).

Final libraries were sequenced at the UTMB Next-Generation Sequencing core using a 300-cycle low-volume flowcell of an Illumina MiniSeq (yields ˜8M 2×150 reads). Since the demultiplexing barcode was present in the first 16 nts of the forward (R1) reads, a custom python script was developed that split the output ‘unclassified’ reads into their correct FASTQ bins if the correct barcode was found in these first 16 nts.

Data Analysis. The de-multiplexed data were first processed with fastp to remove Illumina adapter and to be filtered with quality control (-a AGATCGGAAGAGC-1 30 (SEQ ID NO: 33). This was followed by ViReMa analysis which detects hybrid reads that mapped to both virus (HIV) and host (hg19) genomes (--Seed 30-MicroInDel 5-BackSplice_limit 21-FuzzEntry-Defuzz 0-X 3). This resulted in an output file named “Virus-to-Host Recombinations.BEDPE” which details all the discovered Virus-Host fusion events and the loci of respective genomes. The same bioinformatic analyses were conducted on both Nested-ClickSeq method and regular ClickSeq method. The number of revealed insertion sites was normalized to per one million of sequenced reads (after fastp filtering).

FIG. 5 is a graph that summarizes the results obtained from positive samples. From positive samples (J-Lat cell line with documented HIV pro-virus genome integrations), nested-ClickSeq successfully revealed 1821˜12766 virus integration events per one million of reads, from 4 primers. In comparison, the same primers could only detect 2-123 integration events per one million of reads, with regular ClickSeq. The only exceptions are with primer 5-1 and primer 5-2, which were unable to discover integration events with nested-ClickSeq but discovered 3 and 44 events (per one million of reads) with regular ClickSeq. By way of explanation but not a limitation of the present invention, this may be due to the discrepancy of usage of HIV 5′ and 3′ LTRs during host genome insertion. Further improvement of nested-ClickSeq can be improved with cDNA length control (e.g., by using a higher ratio of AzNTP:dNTP to shorten the cDNA length, which brings virus LTRs closer to the 5′ terminus of cDNA) and/or longer sequencing platforms (e.g., 300 cycle flowcell).

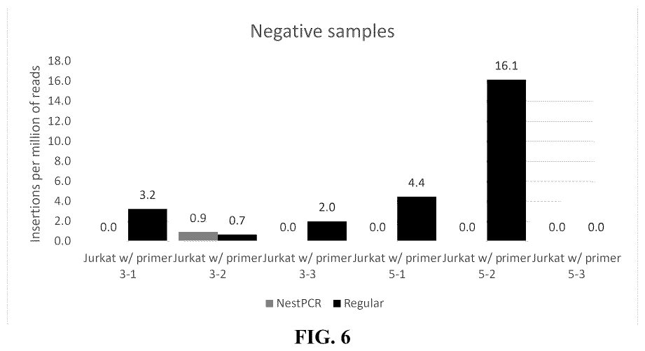

FIG. 6 is a graph that summarizes the results obtained from negative samples. From negative samples (Jurkat cell line without HIV pro-virus insertion), nested-ClickSeq also exhibited equal or better specificity than regular ClickSeq. With nested-ClickSeq, only one primer (3-2) reported 0.9 false positives per one million of reads, whereas all others reported 0 false positive. This is also contrasted by regular ClickSeq which reported 0-16.1 false positives per one million of reads.

In summary, nested-ClickSeq out-performed regular ClickSeq to discover HIV genome integrations (by 37-6,000 fold), and remained excellent in preventing false positives.

The inventors devised, developed and reduced to practical a highly streamlined method for the sequencing of provirus/transgene integration sites in DNA samples. By exploiting the ClickSeq platform for NGS, the inventors took advantage of the numerous features (commercial and scientific) inherent to that platform.

Since ‘Tiled-ClickSeq’ only uses Tiled-primers in the RT-step, the use of template-specific primers in the final PCR step is novel. Presently, all other available platforms that perform something similar also require additional steps after this PCR to ‘complete’ the library, thus making them inferior to the current approach. The present invention can be used in conjunction with random-primed RT-reaction to generate libraries in certain situations where the abundance of the viral target is low or limiting. Moreover, the approach of the present invention is useful in clinical sequencing and targeted surveillance of pathogens. The simplicity of the approach makes it highly amenable for automation in clinical pathology settings and is thus amenable to widespread use.

The combination of this strategy together with using a template-specific primer in the RT-step yields a highly specific and sensitive ‘Nested-ClickSeq’. The sequencing of transgene/provirus integration sites has immense commercial potential as a simple service or kit and is also amenable to automation. Provirus sequencing is routine and important in clinical settings (e.g., HIV treatment). Another market is the validation of transgene insert in gene therapy-based biotechnologies. Here, sensitive and accurate methods are critical to evaluate the success and safety of gene therapies as well as to characterize the off-target integrations events that can be caused by aberration of CRIPSR-based gene therapies.

### LISTING OF EMBODIMENTS

Embodiment 1. A method for cDNA synthesis of a target nucleic acid sequence:
contacting a target nucleic acid of RNA or DNA with one or more first primers comprising one or more first sequences complementary to a target of interest;
performing a reverse transcription reaction with a target nucleic acid, the one or more first primers and one of more terminating nucleotides selected from modified-deoxyGTP, modified-deoxyTTP, modified-deoxyUTP, modified-deoxyCTP and modified-deoxyATP, dNTPs and a reverse transcriptase to form azido-terminated cDNAs of various lengths;
chemically ligating a functionalized 5′ first adaptor to the terminated cDNAs; and
amplifying the chemically-ligated terminated cDNA into an amplification product using one or more second primers comprising one or more second sequences complementary to the target of interest and optionally a second adaptor sequence, wherein the one or more second primers comprises a sequence nested between the one or more first primers and a 3′ end of the azido-terminated cDNA molecule, wherein the cDNA comprises sequences from the target of interest.

Embodiment 2. The method of embodiment 1, wherein the target nucleic acid is a host sequence and further comprising sequencing the host sequence to determine a location of the target of interest.

Embodiment 3. The method of embodiments 1 or 2, wherein the cDNA further comprises additional sequences downstream of the one or more first primers.

Embodiment 4. The method of any one of embodiments 1 to 3, wherein the target of interest is selected from natural or synthetic nucleic acid sequences, either RNA or DNA, such as viral, bacterial, fungal, archaeal, plant or metazoan nucleic acid sequences, including their genome sequences or transcripts thereof, which potentially bear mutations, translocations, fusions, insertions, deletions, integrated proviruses, transposons or transgenes, or be integrated into another nucleic acid.

Embodiment 5. The method of any one of embodiments 1 to 4, wherein the modified-deoxyGTP, modified-deoxyCTP, modified-deoxyTTP, modified-deoxyUTP, and modified-deoxyATP are 2′- or 3′-azido-nucleotides selected from azido-GTP (AzGTP), 2′- or 3′-azido-CTP (AzCTP), 2′- or 3′-azido-ATP (AzATP), 2′- or 3′-azido-TTP (AzUTP), and 2′- or 3′-azido-TTP (AzTTP), or propargyl-GTP, propargyl-TTP, propargyl-UTP, propargyl-CTP, or propargyl-ATP.

Embodiment 6. The method of any one of embodiments 1 to 5, wherein a ratio of the 2′- or 3′-azido-nucleotides (e.g. AzGTP, AzCTP, AzTTP, AzUTP, and AzATP) to dNTPs is 1:1000, 1:900, 1:800, 1:750, 1:700, 1:600, 1:500, 1:400, 1:300, 1:250, 1:200, 1:100, 1:90, 1:80, 1:75, 1:70, 1:60, 1:50, 1:40, 1:30, 1:25, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 1:0.5.

Embodiment 7. The method of any one of embodiments 1 to 6, wherein a ratio of AzTTP or AzUTP:AzGTP:AzCTP:AzATP is w:x:y:z, wherein w is 0.1-10.0, x is 0.1-10.0, y is 0.1-10.0, and z is 0.1-10.0.

Embodiment 8. The method of any one of embodiments 1 to 7, further comprising purifying the cDNA away from the 3′-azido-nucleotides after the reverse transcription and before the amplification step.

Embodiment 9. The method of any one of embodiments 8, further comprising a purification step after the chemical ligation, the amplification, or both, is by column separation, magnetic bead separation, streptavidin magnetic bead wash, precipitation, solute sequestration, or surface immobilization.

Embodiment 10. The method of any one of embodiments 1 to 9, further comprising separating the amplification products according to their length, by gel electrophoresis, polyacrylamide gel electrophoresis, capillary electrophoresis, pulsed-field electrophoresis, agarose gel electrophoresis, PAGE, Solid Phase Reversible Immobilization (SPRI) size fractionation, selective precipitation, or pulsed-field capillary electrophoresis.

Embodiment 11. The method of any one of embodiments 1 to 10, wherein the step of chemically ligating is defined further as click-ligating an alkyne-functionalized 5′ adaptor to the azido-terminated cDNA or an azide-functionalized 5′ adaptor to a propargyl-terminated cDNA is defined further as taking place in a buffered solution comprising: a solvent; with or without one or more metal catalysts selected from copper and ruthenium; a chelating ligand; and an accelerant.

Embodiment 12. The method of any one of embodiments 1 to 11, further comprising purifying the chemically ligated-cDNA-adaptor away from unligated adaptors before an amplification step.

Embodiment 13. The method of any one of embodiment 12, wherein the purification step is by column separation, magnetic bead separation, selective precipitation, surface immobilization, or streptavidin magnetic bead wash.

Embodiment 14. The method of any one of embodiments 1 to 13, wherein the reverse transcription is performed by a reverse transcriptase (RT) derived from Avian Myeloblastosis Virus Reverse Transcriptase, Respiratory Syncytial Virus Reverse Transcriptase, Moloney Murine Leukemia Virus Reverse Transcriptase, Human Immunodeficiency Virus Reverse Transcriptase, Equine Infectious Anemia Virus Reverse Transcriptase, Rous-Associated Virus 2 Reverse Transcriptase, Avian Sarcoma Leukosis Virus Reverse Transcriptase, RNaseH (−) Reverse Transcriptase, SuperScript II Reverse Transcriptase, SuperScript III Reverse Transcriptase, SuperScript IV Reverse Transcriptase, thermostable group II intron reverse transcriptases (TGIRT), Therminator DNA Polymerase, or ThermoScript Reverse Transcriptase, wherein an RNase H activity of these RTs is present, reduced or not present.

Embodiment 15. The method of any one of embodiments 1 to 14, further comprising determining an identity or sequence of the amplification products by an automated process on a chip, Sanger sequencing, Maxam-Gilbert sequencing, dye terminator sequencing, sequencing by synthesis, pyrosequencing, microarray hybridization, next-generation sequencing methods, next-next-generation sequencing, ion semiconductor sequencing, polony sequencing, sequencing by ligation, DNA nanoball sequencing, or single molecule sequencing.

Embodiment 16. The method of any one of embodiments 1 to 15, wherein the genomic nucleic acid is from a biological fluid, biopsy, cells, or tissue.

Embodiment 17. The method of any one of embodiments 1 to 16, wherein the first, the second, and one or more additional primers are present in the same orientation, direction, or strandedness.

Embodiment 18. The method of any one of embodiments 1 to 17, wherein a selectivity of the reverse transcription, the amplification, or both, is increased by using trehalose, betaine, tetramethylammonium chloride, tetramethylammonium oxalate, formamide and oligo-blockers, or dimethylsulfoxide during a polymerase chain reaction, to reduce an occurrence of mispriming.

Embodiment 19. The method of any one of embodiments 1 to 18, wherein a DNA polymerase used for the amplifying step is Taq DNA polymerase, Tfl DNA polymerase, a Taq DNA polymerase, a Klenow fragment, Sequenase or Klentaq an enzyme with proof reading activity, preferably selected from the PFU, Ultma, Vent, Deep Vent, PWO, or Tli polymerase.

Embodiment 20. The method of any one of embodiments 1 to 19, further comprising purifying a PCR product from the step of amplifying the clicked-cDNA with a column separation, magnetic bead separation, selective precipitation, surface immobilization or streptavidin magnetic bead wash.

Embodiment 21. The method of any one of embodiments 1 to 20, wherein an alkyne-functionalized, or azide-functionalized, 5′ adaptor comprises all nucleotides NNNNNN, NO-24, a, Unique Molecular Index, Barcoding Index, semi-random primers, or a specific template primer sequence, or the adapter comprises a unique sequence.

Embodiment 22. The method of any one of embodiments 1 to 21, wherein the terminating deoxynucleotides contain a chemically reactive functional group at either the 3′ or 2′ site of the ribose ring selected from azido-nucleotides (AzGTP, AzCTP, AzTTP, AzUTP and AzATP), propargyl-nucleotides (propargyl-GTP, propargyl-CTP, propargyl-TTP, propargyl-UTP, and propargyl-ATP), amino-nucleotides (AmGTP, AmCTP, AmTTP, AmUTP, and AmATP), or halogenated nucleotides (Hal-GTP, Hal-CTP, Hal-TTP, Hal-UTP, and Hal-ATP).

Embodiment 23. A kit for nested-ClickSeq comprising:
one or more vials comprising:
one or more first primers comprising one or more first sequences complementary to a target of interest;
terminating nucleotides of modified-deoxyGTP, modified-deoxyTTP, modified-deoxyUTP, modified-deoxyCTP, and modified-deoxyATP, and dNTPs;
one or more vials comprising a reverse transcriptase;
a cDNA fragment isolating kit;
one or more vials comprising components for chemically ligating a functionalized 5′ first adaptor to the cDNA;
a DNA amplification kit for amplifying the chemically-ligated cDNA into an amplification product;
a functionalized 5′ first adaptor for chemically ligating to a terminated cDNAs; and
one or more second primers comprising one or more second sequences complementary to the target of interest and optionally a second adaptor sequence, wherein the one or more second primers comprises a sequence nested between the one or more first primers and a 3′ end of the azido-terminated cDNA molecule, wherein the cDNA comprises sequences from the target of interest, wherein the one or more second primers amplify a chemically-ligated terminated cDNA; and
instructions for amplification of a transposable element inserted in a host genome without fragmentation or enzymatic ligation.

Embodiment 24. The kit of embodiment 23, wherein the terminating modified-deoxyGTP, modified-deoxyCTP, modified-deoxyTTP, modified-deoxyUTP, and modified-deoxyATP are 2′- or 3′-azido-nucleotides selected from azido-GTP (AzGTP), 2′- or 3′-azido-CTP (AzCTP), 2′- or 3′-azido-ATP (AzATP), 2′- or 3′-azido-TTP (AzTTP), and 2′- or 3′-azido-UTP (AzUTP), or propargyl-GTP, propargyl-TTP, propargyl-UTP, propargyl-CTP, or propargyl-ATP.

Embodiment 25. The kit of embodiments 24, wherein an alkyne or azide modified oligo ‘click’ reaction is a hexanyl-oligo or azide-oligo.

Embodiment 26. The kit of any one of embodiments 24 or 25, wherein a ratio of the 2′- or 3′-azido-nucleotides (AzGTP, AzCTP, AzTTP, and AzATP) to dNTPs is 1:1000, 1:900, 1:800, 1:750, 1:700, 1:600, 1:500, 1:400, 1:300, 1:250, 1:200, 1:100, 1:90, 1:80, 1:75, 1:70, 1:60, 1:50, 1:40, 1:30, 1:25, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 1:0.5.

Embodiment 27. The kit of any one of embodiments 24 to 26, wherein a ratio of AzTTP or AzUTP:AzGTP:AzCTP:AzATP is w:x:y:z, wherein w is 0.1-10.0, x is 0.1-10.0, y is 0.1-10.0, and z is 0.1-10.0.

Embodiment 28. The kit of any one of embodiments 23 to 17, further comprising a cDNA purification kit for purifying a cDNA away from 2′ or 3′-azido-nucleotides after reverse transcription and before the amplifying step selected from a column separation kit, magnetic bead separation kit, streptavidin magnetic bead kit, precipitation, solute sequestration, or surface immobilization.

Embodiment 29. The kit of any one of embodiments 23 to 28, further comprising a clicked-cDNA-adaptor purification kit for separating clicked-cDNA-adaptors away from unligated alkyne-functionalized 5′ adaptors before the amplifying step selected from a column separation kit, magnetic bead separation kit, streptavidin magnetic bead kit, precipitation, solute sequestration, or surface immobilization.

Embodiment 30. The kit of any one of embodiments 23 to 19, wherein the click-ligating components comprise: an alkyne-functionalized 5′ adaptor to the azido-terminated cDNA or an azido-terminated cDNA; a buffered solution comprising: a solvent mix comprising DMSO, water, and ethanol; metal catalysts selected from copper and ruthenium; a chelating ligand; and an accelerant.

Embodiment 31. The kit of any one of embodiments 23 to 30, wherein the reverse transcriptase (RT) is an RT derived from Avian Myeloblastosis Virus Reverse Transcriptase, Respiratory Syncytial Virus Reverse Transcriptase, Moloney Murine Leukemia Virus Reverse Transcriptase, Human Immunodeficiency Virus Reverse Transcriptase, Equine Infectious Anemia Virus Reverse Transcriptase, Rous-Associated Virus 2 Reverse Transcriptase, Avian Sarcoma Leukosis Virus Reverse Transcriptase, RNaseH (−) Reverse Transcriptase, SuperScript II Reverse Transcriptase, SuperScript III Reverse Transcriptase, SuperScript IV Reverse Transcriptase, thermostable group II intron reverse transcriptases (TGIRT), Therminator DNA Polymerase, or ThermoScript Reverse Transcriptase, wherein an RNase H activity of the RTs is present, reduced or not present.

Embodiment 32. The kit of any one of embodiments 23 to 31, wherein the target of interest is selected from natural or synthetic nucleic acid sequences, either RNA or DNA, such as viral, bacterial, fungal, archaeal, plant or metazoan nucleic acid sequences, including their genome sequences or transcripts thereof, which potentially bear mutations, translocations, fusions, insertions, deletions, integrated proviruses, transposons or transgenes, or be integrated into another nucleic acid.

Embodiment 33. The kit of any one of embodiments 23 to 32, wherein a selectivity of the reverse transcription and/or amplification with a polymerase chain reaction or loop-mediated isothermal amplification, is increased by using trehalose, betaine, tetramethylammonium chloride, tetramethylammonium oxalate, formamide and oligo-blockers, or dimethylsulfoxide during the polymerase chain reaction, to reduce mispriming.

Embodiment 34. The kit of any one of embodiments 23 to 33, further comprising a sequencing kit determining an identity or sequence of the amplification products by an automated process on a chip, Sanger sequencing, Maxam-Gilbert sequencing, dye terminator sequencing, sequencing by synthesis, pyrosequencing, microarray hybridization, next-generation sequencing methods, next-next-generation sequencing, ion semiconductor sequencing, polony sequencing, sequencing by ligation, DNA nanoball sequencing, or single molecule sequencing.

Embodiment 35. The kit of any one of embodiments 23 to 34, wherein a DNA polymerase used for the amplifying reaction is Taq DNA polymerase, Tfl DNA polymerase, Taq DNA polymerase, Klenow fragment, Sequenase or Klentaq with proof reading activity selected from PFU, Ultma, Vent, Deep Vent, PWO, or Tli polymerases.

Embodiment 36. The kit of any one of embodiments 23 to 35, further comprising a kit for purifying a PCR product from the step of amplifying a clicked-nested-cDNA step with a column separation, magnetic bead separation, selective precipitation, surface immobilization or streptavidin magnetic bead wash.

Embodiment 37. The kit of any one of embodiments 23 to 36, wherein the alkyne-functionalized 5′ adaptor comprises all nucleotides NNNNNN, N₀₋₁₂, a Unique Molecular Index, Barcoding Index, semi-random primers, or a specific template primer sequence, or the adapter comprises a unique sequence.

Embodiment 38. The kit of any one of embodiments 23 to 37, wherein the first, the second, and optionally one or more additional nested primers are present in a same orientation, direction, or strandedness.

Embodiment 39. A method for determining a location of a target nucleic acid sequence:
contacting a genomic DNA with one or more first primer comprising a first sequence complementary to a target nucleic acid sequence;
performing a reverse transcription reaction with a genomic nucleic acid, the one or more first primers and one of more terminating nucleotides selected from modified-deoxyGTP, modified-deoxyCTP, modified-deoxyTTP, modified-deoxyATP, dNTPs, and a reverse transcriptase to form terminated cDNAs of various lengths;
chemically ligating a functionalized 5′ first adaptor to the terminated cDNAs;
amplifying the chemically-ligated terminated cDNA into an amplification product using one or more second primers comprising to a second sequence complementary to the target of interest, wherein the one or more second primers comprises a sequence nested between the one or more first primers and a 3′ end of the azido-terminated cDNA molecule, wherein the cDNA comprises sequences from the target of interest; and
sequencing the host sequences and comparing to a genome to determine a location of an insertion of the target of interest.

It is contemplated that any aspects of the disclosure discussed in this specification can be implemented with respect to any method, kit, reagent, or composition of the disclosure, and vice versa. Furthermore, compositions of the disclosure can be used to achieve methods of the disclosure.

It will be understood that particular aspects described herein are shown by way of illustration and not as limitations of the disclosure. The principal features of this disclosure can be employed in various aspects without departing from the scope of the disclosure. Those skilled in the art will recognize, or be able to ascertain using no more than routine experimentation, numerous equivalents to the specific procedures described herein. Such equivalents are considered to be within the scope of this disclosure and are covered by the claims.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In aspects of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the disclosure(s) set out in any claims that may issue from this disclosure. Specifically, and by way of example, although the headings refer to a “Field of Invention,” such claims should not be limited by the language under this heading to describe the so-called technical field. Further, a description of technology in the “Background” section is not to be construed as an admission that technology is prior art to any disclosure(s) in this disclosure. Neither is the “Summary” to be considered a characterization of the disclosure(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure but should not be constrained by the headings set forth herein.

All of the compositions and/or methods disclosed and claimed herein can be made and executed without undue experimentation in light of the present disclosure. While the compositions and methods of this disclosure have been described in terms of preferred aspects, it will be apparent to those of skill in the art that variations may be applied to the compositions and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

What is claimed is:

1. A method for cDNA synthesis of a target nucleic acid sequence:
contacting a target nucleic acid of RNA or DNA with one or more first primers comprising one or more first sequences complementary to a target of interest;
performing a reverse transcription reaction with a target nucleic acid, the one or more first primers and one of more terminating nucleotides selected from modified-deoxyGTP, modified-deoxyTTP, modified-deoxyUTP, modified-deoxyCTP and modified-deoxyATP, dNTPs and a reverse transcriptase to form azido-terminated cDNAs of various lengths;
chemically ligating a functionalized 5′ first adaptor to the terminated cDNAs; and
amplifying the chemically-ligated terminated cDNA into an amplification product using one or more second primers comprising one or more second sequences complementary to the target of interest and optionally a second adaptor sequence, wherein the one or more second primers comprises a sequence nested between the one or more first primers and a 3′ end of the azido-terminated cDNA molecule, wherein the cDNA comprises sequences from the target of interest.

2. The method of claim 1, wherein the target nucleic acid is a host sequence and further comprising sequencing the host sequence to determine a location of the target of interest.

3. The method of claim 1, wherein the cDNA further comprises additional sequences downstream of the one or more first primers.

4. The method of claim 1, wherein the target of interest is selected from natural or synthetic nucleic acid sequences, either RNA or DNA, such as viral, bacterial, fungal, archaeal, plant or metazoan nucleic acid sequences, including their genome sequences or transcripts thereof, suspected of comprising at least one of: mutations, translocations, fusions, insertions, deletions, integrated proviruses, transposons or transgenes, or integration into another nucleic acid.

5. The method of claim 1, wherein at least one of:
the modified-deoxyGTP, modified-deoxyCTP, modified-deoxyTTP, modified-deoxyUTP, and modified-deoxyATP are 2′- or 3′-azido-nucleotides selected from azido-GTP (AzGTP), 2′- or 3′-azido-CTP (AzCTP), 2′- or 3′-azido-ATP (AzATP), 2′- or 3′-azido-TTP (AzUTP), and 2′- or 3′-azido-TTP (AzTTP), or propargyl-GTP, propargyl-TTP, propargyl-UTP, propargyl-CTP, or propargyl-ATP; a ratio of the 2′- or 3′-azido-nucleotides (e.g. AzGTP, AzCTP, AzTTP, AzUTP, and AzATP) to dNTPs is 1:1000, 1:900, 1:800, 1:750, 1:700, 1:600, 1:500, 1:400, 1:300, 1:250, 1:200, 1:100, 1:90, 1:80, 1:75, 1:70, 1:60, 1:50, 1:40, 1:30, 1:25, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 1:0.5;
a ratio of AzTTP or AzUTP:AzGTP:AzCTP:AzATP is w:x:y:z, wherein w is 0.1-10.0, x is 0.1-10.0, y is 0.1-10.0, and z is 0.1-10.0; or
the terminating deoxynucleotides contain a chemically reactive functional group at either the 3′ or 2′ site of a ribose ring selected from azido-nucleotides (AzGTP, AzCTP, AzTTP, AzUTP and AzATP), propargyl-nucleotides (propargyl-GTP, propargyl-CTP, propargyl-TTP, propargyl-UTP, and propargyl-ATP), amino-nucleotides (AmGTP, AmCTP, AmTTP, AmUTP, and AmATP), or halogenated nucleotides (Hal-GTP, Hal-CTP, Hal-TTP, Hal-UTP, and Hal-ATP).

6. The method of claim 1, further comprising at least one of:
purifying the cDNA away from the 3′-azido-nucleotides after the reverse transcription and before the amplification step;
separating the amplification products according to their length, by gel electrophoresis, polyacrylamide gel electrophoresis, capillary electrophoresis, pulsed-field electrophoresis, agarose gel electrophoresis, PAGE, Solid Phase Reversible Immobilization (SPRI) size fractionation, selective precipitation, or pulsed-field capillary electrophoresis;
chemically ligating is defined further as click-ligating an alkyne-functionalized 5′ adaptor to the azido-terminated cDNA or an azide-functionalized 5′ adaptor to a propargyl-terminated cDNA is defined further as taking place in a buffered solution comprising: a solvent; with or without one or more metal catalysts selected from copper and ruthenium; a chelating ligand; and an accelerant; purifying the chemically ligated-cDNA-adaptor away from unligated adaptors before an amplification step; or
determining an identity or sequence of the amplification products by an automated process on a chip, Sanger sequencing, Maxam-Gilbert sequencing, dye terminator sequencing, sequencing by synthesis, pyrosequencing, microarray hybridization, next-generation sequencing methods, next-next-generation sequencing, ion semiconductor sequencing, polony sequencing, sequencing by ligation, DNA nanoball sequencing, or single molecule sequencing.

7. The method of claim 6, wherein the purification step is a purification step after the chemical ligation, the amplification, or both, is by column separation, magnetic bead separation, streptavidin magnetic bead wash, precipitation, solute sequestration, or surface immobilization, or the purification step is by column separation, magnetic bead separation, selective precipitation, surface immobilization, or streptavidin magnetic bead wash.

8. The method of claim 1, wherein at least one of:
the reverse transcription is performed by a reverse transcriptase (RT) derived from Avian Myeloblastosis Virus Reverse Transcriptase, Respiratory Syncytial Virus Reverse Transcriptase, Moloney Murine Leukemia Virus Reverse Transcriptase, Human Immunodeficiency Virus Reverse Transcriptase, Equine Infectious Anemia Virus Reverse Transcriptase, Rous-Associated Virus 2 Reverse Transcriptase, Avian Sarcoma Leukosis Virus Reverse Transcriptase, RNaseH (−) Reverse Transcriptase, SuperScript II Reverse Transcriptase, SuperScript III Reverse Transcriptase, SuperScript IV Reverse Transcriptase, thermostable group II intron reverse transcriptases (TGIRT), Therminator DNA Polymerase, or ThermoScript Reverse Transcriptase, wherein an RNase H activity of these RTs is present, reduced or not present; or
a DNA polymerase used for the amplifying step is Taq DNA polymerase, Tfl DNA polymerase, a Taq DNA polymerase, a Klenow fragment, Sequenase or Klentaq an enzyme with proof reading activity, preferably selected from the PFU, Ultma, Vent, Deep Vent, PWO, or Tli polymerase.

9. The method of claim 1, wherein the target nucleic acid is a genomic nucleic acid is from a biological fluid, biopsy, cells, or tissue.

10. The method of claim 1, wherein the first, the second, and one or more additional primers are present in the same orientation, direction, or strandedness.

11. The method of claim 1, wherein a selectivity of the reverse transcription, the amplification, or both, is increased by using trehalose, betaine, tetramethylammonium chloride, tetramethylammonium oxalate, formamide and oligo-blockers, or dimethylsulfoxide during a polymerase chain reaction, to reduce an occurrence of mispriming.

12. The method of claim 1, further comprising purifying a PCR product from the step of amplifying the clicked-cDNA with a column separation, magnetic bead separation, selective precipitation, surface immobilization or streptavidin magnetic bead wash.

13. The method of claim 1, wherein an alkyne-functionalized, or azide-functionalized, 5′ adaptor comprises all nucleotides NNNNNN, N₀₋₂₄, a, Unique Molecular Index, Barcoding Index, semi-random primers, or a specific template primer sequence, or the adapter comprises a unique sequence.

14. A kit for nested-ClickSeq comprising:
one or more vials comprising:
one or more first primers comprising one or more first sequences complementary to a target of interest;
terminating nucleotides of modified-deoxyGTP, modified-deoxyTTP, modified-deoxyUTP, modified-deoxyCTP, and modified-deoxyATP, and dNTPs;
one or more vials comprising a reverse transcriptase;
a cDNA fragment isolating kit;
one or more vials comprising components for chemically ligating a functionalized 5′ first adaptor to the cDNA;
a DNA amplification kit for amplifying the chemically-ligated cDNA into an amplification product;
a functionalized 5′ first adaptor for chemically ligating to a terminated cDNAs; and
one or more second primers comprising one or more second sequences complementary to the target of interest and optionally a second adaptor sequence, wherein the one or more second primers comprises a sequence nested between the one or more first primers and a 3′ end of the azido-terminated cDNA molecule, wherein the cDNA comprises sequences from the target of interest, wherein the one or more second primers amplify a chemically-ligated terminated cDNA; and
instructions for amplification of a transposable element inserted in a host genome without fragmentation or enzymatic ligation.

15. The kit of claim 14, wherein at least one of:
the terminating modified-deoxyGTP, modified-deoxyCTP, modified-deoxyTTP,
modified-deoxyUTP, and modified-deoxyATP are 2′- or 3′-azido-nucleotides selected from azido-GTP (AzGTP), 2′- or 3′-azido-CTP (AzCTP), 2′- or 3′-azido-ATP (AzATP), 2′- or 3′-azido-TTP (AzTTP), and 2′- or 3′-azido-UTP (AzUTP), or propargyl-GTP, propargyl-TTP, propargyl-UTP, propargyl-CTP, or propargyl-ATP;
an alkyne or azide modified oligo ‘click’ reaction is a hexanyl-oligo or azide-oligo;
a ratio of the 2′- or 3′-azido-nucleotides (AzGTP, AzCTP, AzTTP, and AzATP) to dNTPs is 1:1000, 1:900, 1:800, 1:750, 1:700, 1:600, 1:500, 1:400, 1:300, 1:250, 1:200, 1:100, 1:90, 1:80, 1:75, 1:70, 1:60, 1:50, 1:40, 1:30, 1:25, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 1:0.5; or
a ratio of AzTTP or AzUTP:AzGTP:AzCTP:AzATP is w:x:y:z, wherein w is 0.1-10.0, x is 0.1-10.0, y is 0.1-10.0, and z is 0.1-10.0.

16. The kit of claim 14, further comprising at least one of:
a cDNA purification kit for purifying a cDNA away from 2′ or 3′-azido-nucleotides after reverse transcription and before the amplifying step selected from a column separation kit, magnetic bead separation kit, streptavidin magnetic bead kit, precipitation, solute sequestration, or surface immobilization;
a clicked-cDNA-adaptor purification kit for separating clicked-cDNA-adaptors away from unligated alkyne-functionalized 5′ adaptors before the amplifying step selected from a column separation kit, magnetic bead separation kit, streptavidin magnetic bead kit, precipitation, solute sequestration, or surface immobilization;
wherein the click-ligating components comprise: an alkyne-functionalized 5′ adaptor to the azido-terminated cDNA or an azido-terminated cDNA; a buffered solution comprising: a solvent mix comprising DMSO, water, and ethanol; metal catalysts selected from copper and ruthenium; a chelating ligand; and an accelerant;
the reverse transcriptase (RT) is an RT derived from Avian Myeloblastosis Virus Reverse Transcriptase, Respiratory Syncytial Virus Reverse Transcriptase, Moloney Murine Leukemia Virus Reverse Transcriptase, Human Immunodeficiency Virus Reverse Transcriptase, Equine Infectious Anemia Virus Reverse Transcriptase, Rous-Associated Virus 2 Reverse Transcriptase, Avian Sarcoma Leukosis Virus Reverse Transcriptase, RNaseH (−) Reverse Transcriptase, SuperScript II Reverse Transcriptase, SuperScript III Reverse Transcriptase, SuperScript IV Reverse Transcriptase, thermostable group II intron reverse transcriptases (TGIRT), Therminator DNA Polymerase, or ThermoScript Reverse Transcriptase, wherein an RNase H activity of the RTs is present, reduced or not present; or
a DNA polymerase used for the amplifying reaction is Taq DNA polymerase, Tfl DNA polymerase, Taq DNA polymerase, Klenow fragment, Sequenase or Klentaq with proof reading activity selected from PFU, Ultma, Vent, Deep Vent, PWO, or Tli polymerases.

17. The kit of claim 14, wherein the target of interest is selected from natural or synthetic nucleic acid sequences, either RNA or DNA, such as viral, bacterial, fungal, archaeal, plant or metazoan nucleic acid sequences, including their genome sequences or transcripts thereof, which potentially bear mutations, translocations, fusions, insertions, deletions, integrated proviruses, transposons or transgenes, or be integrated into another nucleic acid.

18. The kit of claim 14, wherein a selectivity of the reverse transcription and/or amplification with a polymerase chain reaction or loop-mediated isothermal amplification, is increased by using trehalose, betaine, tetramethylammonium chloride, tetramethylammonium oxalate, formamide and oligo-blockers, or dimethylsulfoxide during the polymerase chain reaction, to reduce mispriming.

19. The kit of claim 14, further comprising a sequencing kit determining an identity or sequence of the amplification products by an automated process on a chip, Sanger sequencing, Maxam-Gilbert sequencing, dye terminator sequencing, sequencing by synthesis, pyrosequencing, microarray hybridization, next-generation sequencing methods, next-next-generation sequencing, ion semiconductor sequencing, polony sequencing, sequencing by ligation, DNA nanoball sequencing, or single molecule sequencing.

20. The kit of claim 14, further comprising a kit for purifying a PCR product from the step of amplifying a clicked-nested-cDNA step with a column separation, magnetic bead separation, selective precipitation, surface immobilization or streptavidin magnetic bead wash.

21. The kit of claim 14, wherein the alkyne-functionalized 5′ adaptor comprises all nucleotides NNNNNN, N₀₋₁₂, a Unique Molecular Index, Barcoding Index, semi-random primers, or a specific template primer sequence, or the adapter comprises a unique sequence.

22. The kit of claim 14, wherein the first, the second, and optionally one or more additional nested primers are present in a same orientation, direction, or strandedness.

23. A method for determining a location of a target nucleic acid sequence:
contacting a genomic DNA with one or more first primer comprising a first sequence complementary to a target nucleic acid sequence;
performing a reverse transcription reaction with a genomic nucleic acid, the one or more first primers and one of more terminating nucleotides selected from modified-deoxyGTP, modified-deoxyCTP, modified-deoxyTTP, modified-deoxyATP, dNTPs, and a reverse transcriptase to form terminated cDNAs of various lengths;
chemically ligating a functionalized 5′ first adaptor to the terminated cDNAs;
amplifying the chemically-ligated terminated cDNA into an amplification product using one or more second primers comprising to a second sequence complementary to the target of interest, wherein the one or more second primers comprises a sequence nested between the one or more first primers and a 3′ end of the azido-terminated cDNA molecule, wherein the cDNA comprises sequences from the target of interest; and
sequencing the host sequences and comparing to a genome to determine a location of an insertion of the target of interest.


SEQUENCE LISTING (33 sequences)

SEQ ID NO: 1 - DNA, 19 bp, synthetic construct
  misc_feature 1..19: note = Synthetic: 5-0
  source 1..19: mol_type = other DNA
ccaatcagggaagtagcct

SEQ ID NO: 2 - DNA, 21 bp, synthetic construct
  misc_feature 1..21: note = Synthetic: 3-0
  source 1..21: mol_type = other DNA
tgcccgtctgttgtgtgactc

SEQ ID NO: 3 - DNA, 74 bp, synthetic construct
  misc_feature 1..74: note = Synthetic: Hex_4N-701-4N_UniRevComp
  modified_base 1: mod_base = OTHER; note = 5Hexynyl
  source 1..74: mol_type = other DNA
nnnnccgcggttnnnnagatcggaagagcgtcgtgtagggaaagagtgtagatctcggtggtcgccgtatcatt

SEQ ID NO: 4 - DNA, 74 bp, synthetic construct
  misc_feature 1..74: note = Synthetic: Hex_4N-702-4N_UniRevComp
  modified_base 1: mod_base = OTHER; note = 5Hexynyl
  source 1..74: mol_type = other DNA
nnnnttataaccnnnnagatcggaagagcgtcgtgtagggaaagagtgtagatctcggtggtcgccgtatcatt

SEQ ID NO: 5 - DNA, 74 bp, synthetic construct
  misc_feature 1..74: note = Synthetic: Hex_4N-703-4N_UniRevComp
  modified_base 1: mod_base = OTHER; note = 5Hexynyl
  source 1..74: mol_type = other DNA
nnnnggacttggnnnnagatcggaagagcgtcgtgtagggaaagagtgtagatctcggtggtcgccgtatcatt

SEQ ID NO: 6 - DNA, 74 bp, synthetic construct
  misc_feature 1..74: note = Synthetic: Hex_4N-704-4N_UniRevComp
  modified_base 1: mod_base = OTHER; note = 5Hexynyl
  source 1..74: mol_type = other DNA
nnnnaagtccaannnnagatcggaagagcgtcgtgtagggaaagagtgtagatctcggtggtcgccgtatcatt

SEQ ID NO: 7 - DNA, 74 bp, synthetic construct
  misc_feature 1..74: note = Synthetic: Hex_4N-705-4N_UniRevComp
  modified_base 1: mod_base = OTHER; note = 5Hexynyl
  source 1..74: mol_type = other DNA
nnnnatccactgnnnnagatcggaagagcgtcgtgtagggaaagagtgtagatctcggtggtcgccgtatcatt

SEQ ID NO: 8 - DNA, 74 bp, synthetic construct
  misc_feature 1..74: note = Synthetic: Hex_4N-706-4N_UniRevComp
  modified_base 1: mod_base = OTHER; note = 5Hexynyl
  source 1..74: mol_type = other DNA
nnnngcttgtcannnnagatcggaagagcgtcgtgtagggaaagagtgtagatctcggtggtcgccgtatcatt

SEQ ID NO: 9 - DNA, 74 bp, synthetic construct
  misc_feature 1..74: note = Synthetic: Hex_4N-707-4N_UniRevComp
  modified_base 1: mod_base = OTHER; note = 5Hexynyl
  source 1..74: mol_type = other DNA
nnnncaagctagnnnnagatcggaagagcgtcgtgtagggaaagagtgtagatctcggtggtcgccgtatcatt

SEQ ID NO: 10 - DNA, 74 bp, synthetic construct
  misc_feature 1..74: note = Synthetic: Hex_4N-708-4N_UniRevComp
  modified_base 1: mod_base = OTHER; note = 5Hexynyl
  source 1..74: mol_type = other DNA
nnnntggatcgannnnagatcggaagagcgtcgtgtagggaaagagtgtagatctcggtggtcgccgtatcatt

SEQ ID NO: 11 - DNA, 74 bp, synthetic construct
  misc_feature 1..74: note = Synthetic: Hex_4N-709-4N_UniRevComp
  modified_base 1: mod_base = OTHER; note = 5Hexynyl
  source 1..74: mol_type = other DNA
nnnnagttcaggnnnnagatcggaagagcgtcgtgtagggaaagagtgtagatctcggtggtcgccgtatcatt

SEQ ID NO: 12 - DNA, 74 bp, synthetic construct
  misc_feature 1..74: note = Synthetic: Hex_4N-710-4N_UniRevComp
  modified_base 1: mod_base = OTHER; note = 5Hexynyl
  source 1..74: mol_type = other DNA
nnnngacctgaannnnagatcggaagagcgtcgtgtagggaaagagtgtagatctcggtggtcgccgtatcatt

SEQ ID NO: 13 - DNA, 74 bp, synthetic construct
  misc_feature 1..74: note = Synthetic: Hex_4N-711-4N_UniRevComp
  modified_base 1: mod_base = OTHER; note = 5Hexynyl
  source 1..74: mol_type = other DNA
nnnntctctactnnnnagatcggaagagcgtcgtgtagggaaagagtgtagatctcggtggtcgccgtatcatt

SEQ ID NO: 14 - DNA, 74 bp, synthetic construct
  misc_feature 1..74: note = Synthetic: Hex_4N-712-4N_UniRevComp
  modified_base 1: mod_base = OTHER; note = 5Hexynyl
  source 1..74: mol_type = other DNA
nnnnctctcgtcnnnnagatcggaagagcgtcgtgtagggaaagagtgtagatctcggtggtcgccgtatcatt

SEQ ID NO: 15 - DNA, 74 bp, synthetic construct
  misc_feature 1..74: note = Synthetic: Hex_4N-713-4N_UniRevComp
  modified_base 1: mod_base = OTHER; note = 5Hexynyl
  source 1..74: mol_type = other DNA
nnnnccaagtctnnnnagatcggaagagcgtcgtgtagggaaagagtgtagatctcggtggtcgccgtatcatt

SEQ ID NO: 16 - DNA, 74 bp, synthetic construct
  misc_feature 1..74: note = Synthetic: Hex_4N-714-4N_UniRevComp
  modified_base 1: mod_base = OTHER; note = 5Hexynyl
  source 1..74: mol_type = other DNA
nnnnttggactcnnnnagatcggaagagcgtcgtgtagggaaagagtgtagatctcggtggtcgccgtatcatt

SEQ ID NO: 17 - DNA, 74 bp, synthetic construct
  misc_feature 1..74: note = Synthetic: Hex_4N-715-4N_UniRevComp
  modified_base 1: mod_base = OTHER; note = 5Hexynyl
  source 1..74: mol_type = other DNA
nnnnggcttaagnnnnagatcggaagagcgtcgtgtagggaaagagtgtagatctcggtggtcgccgtatcatt

SEQ ID NO: 18 - DNA, 74 bp, synthetic construct
  misc_feature 1..74: note = Synthetic: Hex_4N-716-4N_UniRevComp
  modified_base 1: mod_base = OTHER; note = 5Hexynyl
  source 1..74: mol_type = other DNA
nnnnaatccggannnnagatcggaagagcgtcgtgtagggaaagagtgtagatctcggtggtcgccgtatcatt

SEQ ID NO: 19 - DNA, 74 bp, synthetic construct
  misc_feature 1..74: note = Synthetic: Hex_4N-717-4N_UniRevComp
  modified_base 1: mod_base = OTHER; note = 5Hexynyl
  source 1..74: mol_type = other DNA
nnnntaatacagnnnnagatcggaagagcgtcgtgtagggaaagagtgtagatctcggtggtcgccgtatcatt

SEQ ID NO: 20 - DNA, 74 bp, synthetic construct
  misc_feature 1..74: note = Synthetic: Hex_4N-718-4N_UniRevComp
  modified_base 1: mod_base = OTHER; note = 5Hexynyl
  source 1..74: mol_type = other DNA
nnnncggcgtgannnnagatcggaagagcgtcgtgtagggaaagagtgtagatctcggtggtcgccgtatcatt

SEQ ID NO: 21 - DNA, 74 bp, synthetic construct
  misc_feature 1..74: note = Synthetic: Hex_4N-719-4N_UniRevComp
  modified_base 1: mod_base = OTHER; note = 5Hexynyl
  source 1..74: mol_type = other DNA
nnnnatgtaagtnnnnagatcggaagagcgtcgtgtagggaaagagtgtagatctcggtggtcgccgtatcatt

SEQ ID NO: 22 - DNA, 74 bp, synthetic construct
  misc_feature 1..74: note = Synthetic: Hex_4N-720-4N_UniRevComp
  modified_base 1: mod_base = OTHER; note = 5Hexynyl
  source 1..74: mol_type = other DNA
nnnngcacggacnnnnagatcggaagagcgtcgtgtagggaaagagtgtagatctcggtggtcgccgtatcatt

SEQ ID NO: 23 - DNA, 74 bp, synthetic construct
  misc_feature 1..74: note = Synthetic: Hex_4N-721-4N_UniRevComp
  modified_base 1: mod_base = OTHER; note = 5Hexynyl
  source 1..74: mol_type = other DNA
nnnnggtaccttnnnnagatcggaagagcgtcgtgtagggaaagagtgtagatctcggtggtcgccgtatcatt

SEQ ID NO: 24 - DNA, 74 bp, synthetic construct
  misc_feature 1..74: note = Synthetic: Hex_4N-722-4N_UniRevComp
  modified_base 1: mod_base = OTHER; note = 5Hexynyl
  source 1..74: mol_type = other DNA
nnnnaacgttccnnnnagatcggaagagcgtcgtgtagggaaagagtgtagatctcggtggtcgccgtatcatt

SEQ ID NO: 25 - DNA, 74 bp, synthetic construct
  misc_feature 1..74: note = Synthetic: Hex_4N-723-4N_UniRevComp
  modified_base 1: mod_base = OTHER; note = 5Hexynyl
  source 1..74: mol_type = other DNA
nnnngcagaattnnnnagatcggaagagcgtcgtgtagggaaagagtgtagatctcggtggtcgccgtatcatt

SEQ ID NO: 26 - DNA, 74 bp, synthetic construct
  misc_feature 1..74: note = Synthetic: Hex_4N-724-4N_UniRevComp
  modified_base 1: mod_base = OTHER; note = 5Hexynyl
  source 1..74: mol_type = other DNA
nnnnatgaggccnnnnagatcggaagagcgtcgtgtagggaaagagtgtagatctcggtggtcgccgtatcatt

SEQ ID NO: 27 - DNA, 44 bp, synthetic construct
  misc_feature 1..44: note = Synthetic: 5-1
  source 1..44: mol_type = other DNA
caagcagaagacggcatacgagatatctcttgtcttttttggga

SEQ ID NO: 28 - DNA, 45 bp, synthetic construct
  misc_feature 1..45: note = Synthetic: 5-2
  source 1..45: mol_type = other DNA
caagcagaagacggcatacgagatcacagatcaaggatctcttgt

SEQ ID NO: 29 - DNA, 44 bp, synthetic construct
  misc_feature 1..44: note = Synthetic: 5-3
  source 1..44: mol_type = other DNA
caagcagaagacggcatacgagatgtagatccacagatcaagga

SEQ ID NO: 30 - DNA, 44 bp, synthetic construct
  misc_feature 1..44: note = Synthetic: 3-1
  source 1..44: mol_type = other DNA
caagcagaagacggcatacgagatagacccttttagtcagtgtg

SEQ ID NO: 31 - DNA, 44 bp, synthetic construct
  misc_feature 1..44: note = Synthetic: 3-2
  source 1..44: mol_type = other DNA
caagcagaagacggcatacgagatatccctcagacccttttagt

SEQ ID NO: 32 - DNA, 44 bp, synthetic construct
  misc_feature 1..44: note = Synthetic: 3-3
  source 1..44: mol_type = other DNA
caagcagaagacggcatacgagatggtaactagagatccctcag

SEQ ID NO: 33 - DNA, 13 bp, synthetic construct
  misc_feature 1..13: note = Synthetic: oligonucleotide
  source 1..13: mol_type = other DNA
agatcggaagagc